(19) United States
(12) Patent Application Publication
(10) Pub. No.: US 2021/0057069 A1
(43) Pub. Date: Feb. 25, 2021

(54) METHOD AND DEVICE FOR GENERATING MEDICAL REPORT

(71) Applicant: PING AN TECHNOLOGY (SHENZHEN) CO., LTD., Futian District Shenzhen (CN)

(72) Inventors: Chenyu WANG, Futian District Shenzhen (CN); Jianzong WANG, Futian District Shenzhen (CN); Jing XIAO, Futian District Shenzhen (CN)

(21) Appl. No.: 16/633,707

(22) PCT Filed: Jul. 19, 2018
(86) PCT No.: PCT/CN2018/096266

§ 371 Date: Jan. 24, 2020

(30) Foreign Application Priority Data
May 14, 2018 (CN) 201810456351.1

(51) Int. Cl. G16H 15/00; G16H 30/20; G16H 30/40; G06N 3/04

(52) U.S. Cl. CPC G16H 15/00; G06N 3/049; G16H 30/40; G16H 30/20

(57) ABSTRACT

The preset application is applied to the field of information processing technologies, and a method and a device for generating a medical report are provided. The method includes: receiving a medical image to be recognized; importing the medical image into a preset visual geometry group VGG neural network to acquire a visual feature vector and a keyword sequence of the medical image; importing the visual feature vector and the keyword sequence into a preset diagnostic item recognition model to determine diagnostic items corresponding to the medical image; constructing a paragraph for describing each of the diagnostic items respectively based on a diagnostic item extension model; generating a medical report for the medical image based on the paragraph, the keyword sequence and the diagnostic items.

The present application is a National Stage of PCT Application No. PCT/CN2018/096266 filed on Jul. 19, 2018, which claims priority of Chinese patent application No. 201810456351.1, filed on May 14, 2018 and entitled “a method and a device for generating a medical report”, the contents each of which are incorporated herein by reference in their entity.

### TECHNICAL FIELD

The present application relates to the field of information processing technologies, and particularly to a method and a device for generating a medical report.

### BACKGROUND

With continuous development of medical imaging technologies, a doctor can efficiently determine a patient's symptoms through a medical image, and the diagnosis time is greatly reduced. The doctor will manually fill in a corresponding medical report based on the medical image, so that the patient can better understand his own symptoms. However, in the existing methods for generating a medical report, the symptoms cannot be directly determined from the medical image for a patient and a trainee doctor, and it is required to fill in the medical report depending on a experienced doctor, thereby increasing labor cost for generating the medical report. Moreover, manual filling is provided with relatively low efficiency, which undoubtedly increases treatment time for the patient.

### TECHNICAL PROBLEMS

In view of this, embodiments of the present application provide a method and a device for generating a medical report to solve technical problems that the labor cost for generating the medical report is relatively high and the treatment time for the patient is prolonged in the existing methods for generating a medical report.

### SUMMARY

A first aspect of embodiments of the present application provides a method for generating a medical report, which includes:

receiving a medical image to be recognized;

importing the medical image into a preset visual geometry group VGG neural network to acquire a visual feature vector and a keyword sequence of the medical image;

importing the visual feature vector and the keyword sequence into a preset diagnostic item recognition model to determine diagnostic items corresponding to the medical image;

constructing a paragraph for describing each of the diagnostic items respectively based on a diagnostic item extension model;

generating a medical report for the medical image based on the paragraph, the keyword sequence and the diagnostic items.

### BENEFICIAL EFFECTS

In the embodiments of the present application, a visual feature vector and a keyword sequence corresponding to the medical image is determined by importing the medical image into a preset VGG neural network, the visual feature vector is used to characterize the image features of the medical image containing symptoms, and the keyword sequence is used to determine the type of the symptoms contained in the medical image. The above two parameters are imported into a diagnostic item recognition model to determine diagnosis items included in the medical image, and a phrase and a sentence for relevant description for each diagnostic item are filled in so as to form a paragraph corresponding to the diagnostic item, and finally the medical report of the medical image is acquired based on the paragraph corresponding to each diagnosis item. Compared with the existing methods for generating a medical report, there is no need for a doctor to fill in manually in the embodiments of the present application, and the corresponding medical report may automatically output according to the features contained in the medical image, thereby improving the efficiency of generating the medical report, reducing labor cost, and saving treatment time for a patient.

### DESCRIPTION OF THE DRAWINGS

FIG. 1a is a flowchart of implementing the method for generating a medical report according to a first embodiment of the present application.

FIG. 1b is a block diagram of a structure of a VGG neural network according to an embodiment of the present application.

FIG. 1c is a block diagram of a structure of an LSTM neural network according to an embodiment of the present application.

FIG. 2 is a specific flowchart of implementing the method S102 for generating a medical report according to a second embodiment of the present application.

FIG. 3 is a specific flowchart of implementing the method S103 for generating a medical report according to a third embodiment of the present application.

FIG. 4 is a specific flowchart of implementing the method for generating a medical report according to a fourth embodiment of the present application.

FIG. 5 is a specific flowchart of implementing the method for generating a medical report according to a fourth embodiment of the present application.

FIG. 6 is a block diagram of a structure of the device for generating a medical report according to an embodiment of the present application.

FIG. 7 is a schematic diagram of the device for generating a medical report according to another embodiment of the present application.

### EMBODIMENTS OF THE APPLICATION

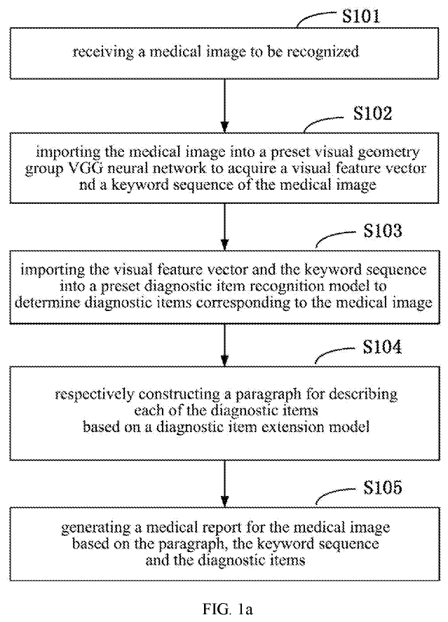

In the embodiments of the present application, the execution subject of the process is the device for generating a medical report. The device for generating a medical report includes, but is not limited to, device for generating a medical report such as a notebook computer, a computer, a server, a tablet computer, and a smart phone etc. FIG. 1a shows a flowchart of implementing the method for generating a medical report according to a first embodiment of the present application, which is described in detail as follows.

At S101, receive a medical image to be recognized.

In this embodiment, the device for generating a medical report may be integrated into a terminal for capturing the medical image. In this case, after the capture terminal completes the capturing operation and generates the medical image for a patient, the medical image may be transmitted to the device for generating a medical report and analyzed to determine the corresponding medical report, thus there is no need to print the medical image to the patient and the doctor, thereby improving the processing efficiency. Of course, the device for generating a medical report may be only connected to a serial port of the capture terminal, and the generated medical image is transmitted through the relevant serial port and interface.

In this embodiment, the device for generating a medical report may operate the medical image acquired by printing through a built-in scanning module, thereby acquiring the computer-readable medical image. Of course, the device for generating a medical report may also receive the medical image sent by a user terminal through a wired communication interface or a wireless communication interface, and then return the medical report acquired by analysis to the user terminal through a corresponding communication channel, thereby achieving the purpose of acquiring the medical report remotely.

In this embodiment, the medical image includes, but is not limited to, an image that a human body is radiated by various types of radiation light such as an X-ray image, a B-mode ultrasound image and the like, and a pathological image such as an anatomical image and an internal organ image of a human body taken based on a microcatheter.

Alternatively, after S101, the generating device may further perform optimization on the medical image through a preset image processing algorithm. The above image processing algorithm includes, but is not limited to, an image processing algorithm such as sharpening processing, binarization processing, noise reduction processing, and grayscale processing etc. In particular, if the medical image is acquired by scanning, the image quality of the acquired medical image may be increased by increasing a scanning resolution, and the medical image may be differentially processed by collecting ambient light intensity at the time of scanning to reduce the impact of the ambient light on the medical image and improve the accuracy of subsequent recognition.

At S102, the medical image is imported into a preset visual geometric group (VGG) neural network to acquire a visual feature vector and a keyword sequence of the medical image.

In this embodiment, the generating device is stored with a Visual Geometry Group (VGG) neural network to process the medical image and extract the visual feature vector and the keyword sequence corresponding to the medical image. Among them, the visual feature vector is used to describe a image feature of an object photographed in the medical image, such as a contour feature, a structure feature, a relative distance between various objects, etc.; the keyword feature is used to characterize the object contained in the medical image and an attribute of the object. For example, if a part captured in the medical image is a chest, the recognized keyword sequence may be: [chest, lung, rib, left lung lobe, right lung lobe, heart], etc. Of course, if there is an abnormal object in a certain part, the abnormal object may be reflected in the keyword sequence. Preferably, there is a one-to-one correspondence between each element of the visual feature vector and each element of the keyword sequence, that is, each element in the visual feature vector is an image feature for describing each keyword in the keyword sequence.

In this embodiment, the VGG neural network may be a VGG19 neural network, since the VGG19 neural network is provided with a strong computing capability in image feature extraction and can extract the visual feature after reducing the dimensionality of the image data including multiple layers through five pooling layers. Moreover, in this embodiment, a fully connected layer is adjusted as a keyword index table, so that the keyword sequence may be output based on the keyword index table. The schematic diagram of the VGG19 may refer to FIG. 1b.

Alternatively, before S102, the generating device may acquire multiple training images to adjust parameters of each of the pooling layers and the fully connected layer in the VGG neural network until an output result converges. That is to say, the training images are used as the input, and the value of each element in the output visual feature vector and the keyword sequence is consistent with a preset value. Preferably, the training images may include not only the medical images, but also other types of images other than the medical images, such as portrait images, static scene images, etc., so that the number of recognizable images is increased in the VGG neural network, thereby improving the accuracy.

At S103, the visual feature vector and the keyword sequence are imported into a preset model for recognizing a diagnosis item, and the diagnosis item corresponding to the medical image is determined.

In this embodiment, shape features corresponding to various objects and the attributes of the objects may be determined by recognizing the keyword sequence and the visual feature vector contained in the medical image, and the above two parameters are imported into the preset model for recognizing the diagnosis item, then the diagnosis item included in the medical image may be determined. The diagnosis item is specifically used to represent a health status of a person being photographed represented by the medical image.

It should be noted that the number of the diagnosis items may be set based on a requirement of an administrator, that is, the number of the diagnosis items included in each of the medical images is the same. In this case, the administrator may also generate a model for recognizing a diagnosis item corresponding to a threshold according to the image type of different medical images. For example, for a chest dialysis image, the model for recognizing the chest diagnosis item may be used; and for an X-ray knee perspective view, the model for recognizing the knee joint diagnosis item may be used. The number of the diagnosis items in all output results of each recognition model is fixed, which means that the preset diagnosis items need to be recognized.

In this embodiment, the model for recognizing the diagnosis item may use a trained LSTM neural network. In this case, the visual feature vector and the keyword sequence may be combined to form a medical feature vector as an input of the LSTM neural network. The layers of the LSTM neural network may match the number of diagnosis items that need to be recognized, that is, each layer of the LSTM neural network corresponds to one diagnosis item. Referring to FIG. 1c, FIG. 1c is a block diagram of a structure of the LSTM neural network according to an embodiment of the present application. The LSTM neural network includes N LSTM layers, and the N LSTM layers correspond to N diagnosis items, where image is the medical feature vector generated based on the visual feature vector and the keyword sequence, S₀·S_{N-1} are parameter values of the various diagnosis items, p₁˜p_{N} are correct probabilities of the various parameter values. When log pᵢ(Sᵢ−1) converges, the parameter value of is used as the parameter value corresponding to the diagnosis item, so as to determine the values of the various diagnosis items in the medical image.

At S104, a paragraph for describing each of the diagnosis items is respectively constructed based on an expanded model of the diagnosis items.

In this embodiment, after determining each of the diagnosis items, the generating device will import the diagnosis items into the expanded model of the diagnosis items, thereby outputting the paragraph describing each of the diagnosis items, such that the patient can intuitively perceive contents of the diagnosis items through the paragraph to improve the readability of the medical report.

Alternatively, the extended model of the diagnosis items may be a hash function, which records corresponding paragraphs when each of the diagnosis items takes different parameter values, and the generating device imports each of the diagnosis items corresponding to the medical image into the hash function respectively, then the paragraphs of the diagnosis items may be determined. In this case, the generating device may determine the paragraphs only through conversion of the hash function, thus the calculation amount is small, thereby improving the efficiency of generating the medical report.

Alternatively, the extended model of the diagnosis items may be an LSTM neural network. In this case, the generating device aggregates all the diagnosis items to form a diagnosis item vector, and uses the diagnosis item vector as an input end of the LSTM neural network. The number of the layers of the LSTM neural network is the same as the number of the diagnosis items, and each layer in the LSTM neural network is used to output the paragraph of one diagnosis item, such that the conversion operation from the diagnosis items to the paragraphs is completed after the output of the multilayer neural network. In the process of generating paragraphs in the above manner, since the input of the LSTM neural network is the diagnosis item vector aggregating each of the diagnosis items and containing information on each of the diagnosis items, one generated paragraph may take into account the impact of other diagnosis items, thereby improving the coherence of among the paragraphs, which in turn improves the readability of the entire medical report. It should be noted that, the specific process of determining the paragraphs through the LSTM neural network is similar to S104, which is not described in detail herein.

At S105, the medical report of the medical image is generated based on the paragraphs, the keyword sequence, and the diagnosis items.

In this embodiment, the medical report of the medical image may be created after the device for generating the medical report determines the diagnosis items included in the medical image, the paragraphs for describing the diagnosis items, and the keywords corresponding to the diagnosis items. It should be noted that, since the paragraphs of the diagnosis items are sufficiently readable, the medical report may be divided into modules based on the diagnosis items, and each of the module is filled in the corresponding paragraph, that is, the medical report visible to the actual user may only contain the contents of the paragraphs and do not directly reflect the diagnosis items and the keywords. Of course, the generating device may associatedly display the diagnosis items, the keywords, and the paragraphs, so that the user may quickly determine the specific contents of the medical report from the short and refined keyword sequence, and determine his/her own health status through the diagnosis items, and then learn about the evaluation of the health status in detail through the paragraphs, and quickly understand the contents of the medical report from different perspectives, thereby improving the readability of the medical report and the efficiency of information acquisition.

Alternatively, the medical report may be attached with the medical images, and the keyword sequence is sequentially marked at the corresponding positions of the medical images, and the diagnosis item and the paragraph Information corresponding to each of the keywords are displayed in a comparison manner by using a marker box, a list, or a column, or the like, such that the user can more intuitively determine the contents of the medical report.

It can be seen from the foregoing that, the method for generating a medical report according to the embodiments of the present application determines a visual feature vector and a keyword sequence corresponding to the medical image by importing the medical image into a preset VGG neural network. The visual feature vector is used to characterize the image features of the medical image containing symptoms, the keyword sequence is used to determine the type of the symptoms contained in the medical image, and the above two parameters are imported into the model for recognizing the diagnosis item to determine the diagnosis item included in the medical image, and to fill in the phrases and sentences for relevant description for each diagnosis item so as to form the paragraph corresponding to the diagnosis item, and finally the medical report of the medical image is acquired based on the paragraph corresponding to each diagnosis item. Compared with the existing methods for generating a medical report, there is no need for a doctor to fill in manually in the embodiments of the present application, and the corresponding medical report may automatically output according to the features contained in the medical image, thereby improving the efficiency of generating the medical report, reducing the labor cost, and saving the treatment time for the patient.

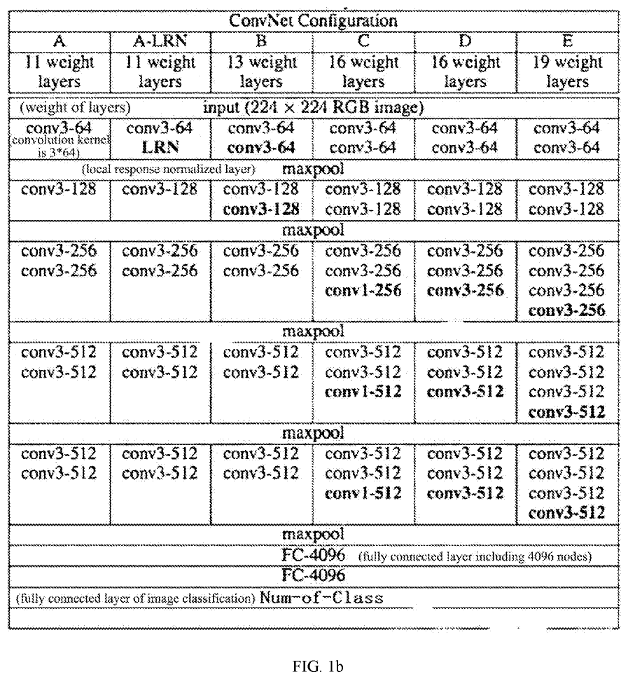

FIG. 2 shows a specific flowchart for implementing the method S102 for generating a medical report according to a second embodiment of the present application. Referring to FIG. 2, compared to the embodiment described in FIG. 1a, in the method for generating a medical report according to this embodiment, S102 includes S1021 to S1024, which is described in details as follows.

At S1021, a pixel matrix of the medical image is constructed based on a pixel value of each of pixel points in the medical image and a position coordinate of each of the pixel values.

In this embodiment, the medical image is composed of a plurality of pixels, and each of the pixels corresponds to one pixel value. Therefore, the pixel values corresponding to the pixels are determined as values of elements corresponding to the coordinates of the pixel points in the pixel matrix based on that the position coordinate of each of the pixels is determined as the position coordinate in the pixel matrix, such that the two-dimensional image may be converted into one pixel matrix.

It should be noted that, if the medical image is a three-primary RGB image, then three pixel matrices may be constructed based on the three layers of the medical image, that is, the R layer corresponds to one pixel matrix, the G layer corresponds to one pixel matrix, and the B layer corresponds to one pixel matrix, and the values of the elements in each of the pixel matrices are 0˜255. Of course, the generating device may also perform grayscale conversion or binarization conversion on the medical image, thereby the multiple layers are fused into one image, so that the number of the constructed pixel matrix is also one. Alternatively, if the medical image is a three-primary RGB image, the pixel matrices corresponding to the multiple layers may be fused to form the pixel matrix corresponding to the medical image. The fusion method may be as follows: the columns in the three pixel matrices are retained and from a one-to-one correspondence to the abscissas of the medical image, the rows of the pixel matrix of the R layer are expanded, and two blank rows are filled between each two row, and each row of the other two pixel matrices is sequentially imported into the expanded various blank rows according to the sequence of the row numbers, thereby constituting a 3M*N pixel matrix, where M is the number of rows of the medical image and N is the number of columns of the medical image.

At S1022, the dimensionality reduction operation is performed on the pixel matrix through the five pooling layers (Maxpools) of the VGG neural network to obtain the visual feature vector.

In this embodiment, the constructed pixel matrix is imported into the five pooling layers of the VGG neural network, and the visual feature vector corresponding to the pixel matrix is generated after five dimensionality reduction operations. It should be noted that, the convolution kernel of the pooling layers may be determined based on the size of the pixel matrix. In this case, the generating device records a correspondence table between the size of the matrix and the convolution kernel, and the generating device will acquire the number of rows and columns of the matrix after constructing the pixel matrix corresponding to the medical image, so as to determine the size of the matrix and look for a size of the convolution kernel corresponding to the size, and the pooling layers in the VGG neural network are adjusted based on the size of the convolution kernel so that the convolution kernel used during the dimensionality reduction operation matches the pixel matrix.

In this embodiment, the VGG neural network includes five pooling layers (Maxpools) for extracting a visual feature and a fully-connected layer for determining a keyword sequence corresponding to the visual feature vector. The medical image is first imported into the five pooling layers, and then the dimensionality-reduced vector is imported into the fully connected layer to output the final keyword sequence. However, in the process of determining the diagnosis item, in addition to acquiring an object to be described and the keyword sequence of the attributes of the object, it is also necessary to determine a visual contour feature for each object. Therefore, the generating device will optimize the initial VGG neural network, and configure a parameter output interface after the five pooling layers to import the intermediate variable (the visual feature vector) for a subsequent operation.

At S1023, the visual feature vector is imported into the fully connected layer of the VGG neural network, and an index sequence corresponding to the visual feature vector is output.

In this embodiment, the generating device will import the visual feature vector to the fully connected layer of the VGG neural network. The fully connected layer records the index number corresponding to each keyword. Since the VGG network is trained, the objects included in the medical image and the attributes of each of the objects may be determined based on the visual feature vector, so that the index sequence corresponding to the visual feature vector may be generated after the operation of the fully connected layer. Because the output of VGG neural network is generally a vector, sequence or matrix composed of numbers, the generating device does not directly output the keyword sequence at S1023, but instead outputs the index sequence corresponding to the keyword sequence. The index sequence contains a plurality of index numbers, and each of the index numbers corresponds to one keyword, so that the keyword sequence corresponding to the medical image may be determined under the condition that the output result only contains numeric characters.

At S1024, the keyword sequence corresponding to the index sequence is determined according to the keyword index table.

In this embodiment, the generating device is stored with the keyword index table, and the keyword index table records the index number corresponding to each of the keywords, so that the generating device may look for the keywords corresponding to the index numbers based on the index number corresponding to each element in the index sequence after determining the index sequence, thereby converting the index sequence into the keyword sequence.

In the embodiments of the present application, the output of the five pooling layers is used as the visual feature vector, and the main features contained in the medical image may be expressed by a one-dimensional vector after the dimensionality reduction operation is performed, thereby reducing the size of the visual feature vector, improving the efficiency of subsequent recognition. Moreover, the output index sequence is converted into the keyword sequence, which reduces the transformation of the VGG model.

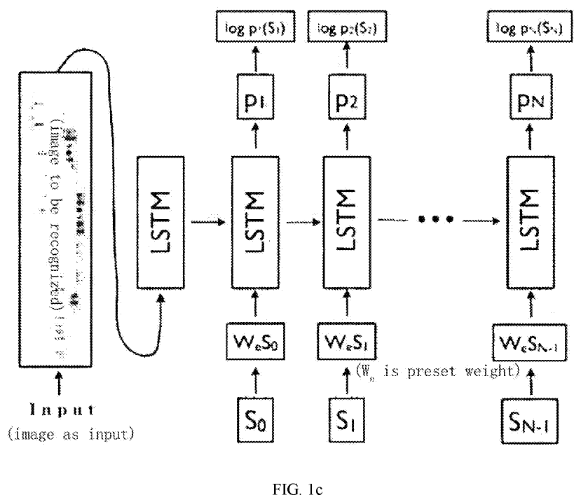

FIG. 3 shows a specific flowchart of implementing the method S103 for generating a medical report according to a third embodiment of the present application. Referring to FIG. 3, compared to the embodiment as shown in FIG. 1a, the method S103 for generating a medical report according to this embodiment includes steps of S1031 to S1033, and the details are described as follows.

At S1031, the keyword feature vector corresponding to the keyword sequence is generated based on the sequence number of each keyword in a preset text corpus.

In this embodiment, the device for generating the medical report is stored with the text corpus that records all keywords. The text corpus will configure the sequence number for response for each keyword, and the generating device may convert the keyword sequence into its corresponding keyword feature vector based on the text corpus. The number of elements contained in the keyword feature vector corresponds to the elements contained in the keyword sequence, and the corresponding sequence number of each keyword in the text corpus is recorded in the keyword feature vector, therefore the sequence including multiple character types including text, English, and numbers may be converted into a kind of sequence including numbers only, thereby improving the operability of the keyword characteristic sequence.

It should be noted that the text corpus may be downloaded through a server and the keywords contained in the text corpus may be updated based on the input manner of the user. For new keywords, a corresponding sequence number is configured for each of the newly added keywords based on the original keywords. For a deleted keyword, all the keywords are adjusted after the sequence number of the keyword is deleted, so that the sequence numbers of the various keywords in the entire text corpus are continuous.

At S1032, the keyword feature vector and the visual feature vector are respectively imported into a preprocessing function to acquire a preprocessed keyword feature vector and a preprocessed visual feature vector. The preprocessing function is specifically as:

$$σ ( {z}_{j} ) = \frac{{e}^{{z}_{j}}}{{Σ}_{i = 1}^{M} {e}^{{z}_{i}}}$$

Where, σ(zⱼ) is the value after the j-th element in the keyword feature vector or in the visual feature vector is preprocessed, zⱼ is the value of the j-th element in the keyword feature vector or in the visual feature vector, M is the number of elements corresponding to the keyword feature vector or the visual feature vector.

In this embodiment, when the position difference of the various keywords in the keyword sequence is relatively large in the text corpus, the numerical difference of the sequence numbers contained in the generated keyword feature vector is then relatively large, which is not conducive to the storage of the keyword feature vector and subsequent processing. Therefore, at S1032, the keyword feature vector is pre-processed to ensure that the values of all elements in the keyword feature sequence are within a preset range, so as to reduce the storage space of the keyword feature vector and reduce the amount of calculation for diagnostic item identification.

For the same reasons, the visual feature vector may also be pre-processed to convert the values of the various elements in the visual feature vector to be within a preset numerical range.

The specific manner of the preprocessing function in this embodiment is as described above. The values of the various elements are accumulated to determine the proportion of each of the elements to the entire vector, and the proportion is used as a parameter of the element after the element is preprocessed, thereby ensuring that the value range of all elements in the visual feature vector and the keyword feature vector is from 0 to 1, which can reduce the storage space for the above two sets of vectors.

At S1033, the preprocessed keyword feature vector and the preprocessed visual feature vector are used as the input of the model of the diagnostic item recognition, and the diagnostic item is output.

In this embodiment, the generating device uses the preprocessed keyword vector and the preprocessed visual feature vector as the input of the model of the diagnostic item recognition. The values of the above two sets of vectors are within a preset range after being processed above, thus the number of bytes allocated for each element is reduced and the size of the entire vector is effectively controlled. When calculation is performed on the model of the diagnostic item recognition, the read operations for invalid digits can also be reduced, which improves the processing efficiency. Moreover, the parameter value of each element in the above vector has not be changed substantially, but has been reduced proportionally, so the diagnostic item can still be determined.

It should be noted that the above recognition model for the diagnostic item may refer to LSTM neural network and the neural network provided in the foregoing embodiments. The specific implementation processes may refer to the foregoing embodiments, and details of which are not described herein again.

In the embodiments of the present application, the keyword sequence and the visual feature vector are preprocessed, thereby improving the generation efficiency of the medical report.

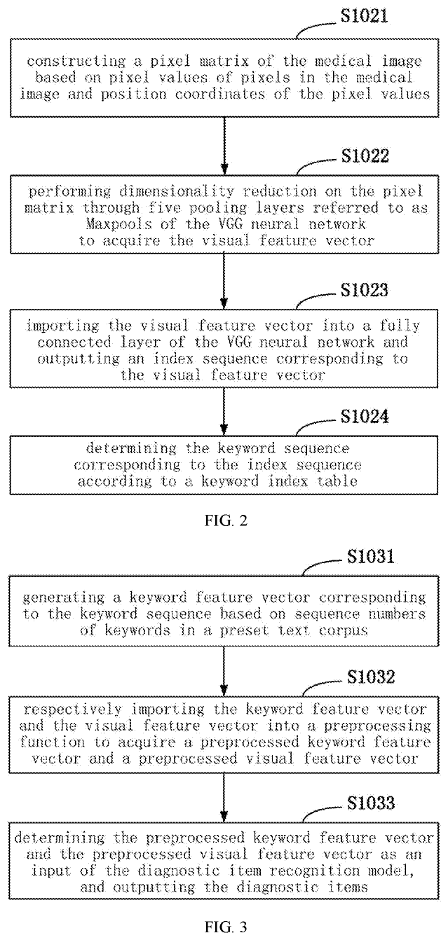

FIG. 4 shows a specific flowchart of implementing the method for generating a medical report according to a fourth embodiment of the present application. Referring to FIG. 4, compared to the embodiments described in FIG. 1a to FIG. 3, the method for generating a medical report according to this embodiment further includes steps of S401 to S403, which are detailed in detail as follows.

Further, before importing the visual feature vector and the keyword sequence into a preset diagnostic item recognition model and determining the diagnostic item corresponding to the medical image, the method further includes the following.

At S401, training visual vectors, training keyword sequences, and training diagnostic items of a plurality of training images are acquired.

In this embodiment, the device for generating a medical report will acquire the training visual vectors, the training keyword sequences, and the training diagnostic items of the plurality of preset training images. Preferably, the number of the training images should be greater than 1000, thereby improving the recognition accuracy of the LSTM neural network. It should be emphasized that the training image may be a historical medical image or other images not limited to medical types, thereby increasing the number of types of recognizable objects for the LSTM neural network.

It should be noted that the format of the training diagnostic item for each training image is the same, that is, the number of items of the training diagnostic item is the same. If part of the training diagnostic items cannot be parsed from any training image due to the shooting angle, the values of the training diagnostic items are empty, thereby ensuring that the meaning of the parameter output from each channel is fixed when training the LSTM neural network, thereby improving the accuracy of LSTM neural network.

At S402, the training visual vector and the training keyword sequence are used as the input of the long short-term LSTM neural network, and the training diagnostic items are used as the output of the LSTM neural network. The learning parameters of the LSTM neural network are adjusted so that the LSTM neural network meets a convergence condition. The convergence condition is as follows:

$${θ}^{*} = {arg max}_{θ} {\sum }_{S t c} log p ( Visual , Keyword \vert Stc ; θ )$$

Where θ* is the adjusted learning parameter, Visual is the training visual vector, Keyword is the training keyword sequence, Stc is the training diagnostic item, p(Visual, Keyword|Stc; θ) represents an output result of a probability value of the training diagnostic item when the training visual vector and the training keyword sequence are imported into the LSTM neural network with the value of the learning parameter is θ, and arg max_{θ}Σ_{Stc} log p(Visual, Keyword|Stc; θ) is the value of the learning parameter when the probability value takes the maximum value.

In this embodiment, the LSTM neural network includes a plurality of neural layers, and each neural layer is provided with a corresponding learning parameter, and it can adapt to different types of inputs and outputs by adjusting the parameter values of the learning parameters. When the learning parameter is set to a certain parameter value, the object images of a plurality of training objects are input to the LSTM neural network, and then the object attributes of the various objects are correspondingly output. The generating device compares the output diagnostic items with the training diagnostic items to determine whether the current output is correct, and acquires the probability value that the output result is correct when the learning parameter takes the parameter value based on the output results of the plurality of training objects. The generating device will adjust the learning parameters, so that the probability value takes the maximum value, which indicates that the LSTM neural network has finished adjustment.

At S403, the adjusted LSTM neural network is used as the diagnostic item recognition model.

In this embodiment, the terminal device uses the LSTM neural network after adjusting the learning parameters as the diagnostic item recognition model, which improves the recognition accuracy for the diagnostic item recognition model.

In the embodiments of the present application, the LSTM neural network is trained by the training objects, and the learning parameters, corresponding to the maximum probability value when the output result is correct, are selected as the parameter values of the learning parameters in the LSTM neural network, thereby improving the accuracy of diagnostic item recognition, and further improving the accuracy of the medical report.

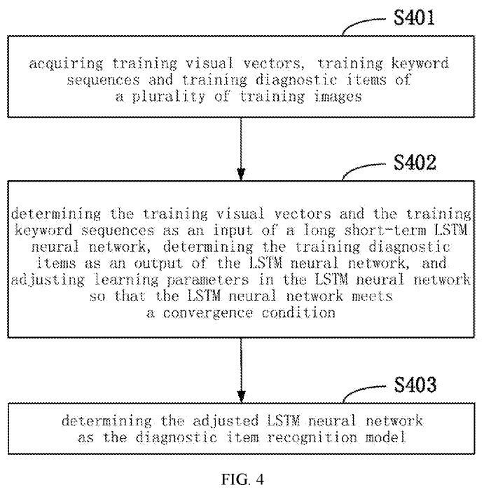

FIG. 5 shows a specific flowchart of implementing the method for generating a medical report according to a fifth embodiment of the present application. Referring to FIG. 5, compared to the embodiment described in FIG. 1a, the method for generating a medical report provided in this embodiment includes steps of S501 to S50, and details of which are described as follows.

At S501, the medical image to be recognized is received.

Since S501 and S101 are implemented in the same manner, its specific parameters may refer to the related description of S101, and the details of which are not described herein again.

At S502, binarization is performed on the medical image to obtain a binarized medical image.

In this embodiment, the generating device will perform binarization on the medical image to make the edges of each object in the medical image more obvious, thereby facilitating the determination of the outline of each object and the internal structure of each object, and facilitating realizing of extraction of the visual feature vector and the keyword sequence.

In this embodiment, the threshold of the binarization may be set according to the user's needs, and the generating device may also determine the threshold of the binarization by determining the type of the medical image and/or the average pixel value of the various pixels in the medical image, thereby improving the display effect of the binarized medical image.

At S503, the boundary of the binarized medical image is identified, and the medical image is divided into a plurality of medical sub-images.

In this embodiment, the generating device may extract the boundaries of each object from the binarized medical image by using a preset boundary identification algorithm, such that the medical image is divided based on the identified boundaries, and separate medical sub-image of each object is acquired. Of course, if several objects are related to each other and their boundaries are overlapping or adjacent, the above-mentioned objects may be integrated into one medical sub-image. By dividing different objects into regions, the influence of a certain object on other objects when performing extraction of the visual features and the keywords is reduced.

Further, the step of importing the medical image into the preset VGG neural network to acquire the visual feature vector and the keyword sequence of the medical image includes the following.

At S504, each of the medical sub-images is imported into the VGG neural network to acquire visual feature components and keyword sub-sequences of the medical sub-images.

In this embodiment, the generating device imports each of the medical sub-images segmented based on the medical image into the VGG neural network, so as to acquire the visual feature component and the keyword sub-sequence corresponding to each of the medical sub-image. The visual feature components are used to represent shape and contour features of the objects in the medical sub-images, and the keyword sub-sequences are used to represent the objects contained in the medical sub-images. Through dividing the medical image and importing them into the VGG neural network, the amount of data in each operation of the VGG neural network can be reduced, thereby greatly reducing processing time and improving output efficiency. Moreover, since the division is based on the boundaries, most of the invalid images in the background region can be effectively deleted, such that the overall data processing amount will be greatly reduced.

At S505, the visual feature vector is generated based on the various visual feature components, and the keyword sequence is formed based on the various keyword sub-sequences.

In this embodiment, the visual feature components of the various medical sub-images are combined to form the visual feature vector of the medical image. Similarly, the keyword sub-sequences of the various medical sub-images are combined to form the keyword sequence of the medical image. It should be noted that during the combination process, the position of the visual feature component of certain medical sub-image in the combined visual feature vector corresponds to the position of the keyword sub-sequence of the medical sub-image in the combined keyword sequence, so as to maintain the relationship between the visual feature component and the keyword sub-sequence.

At S506, the visual feature vector and the keyword sequence are imported into the preset diagnostic item recognition model, and the diagnostic items corresponding to the medical image are determined.

At S507, the paragraphs for describing each of the diagnostic items are respectively constructed based on the diagnostic item extension model.

At S508, the medical report of the medical image is generated based on the paragraphs, the keyword sequence, and the diagnosis items.

Since S506˜S508 are implemented in the same way as S103˜S105, the specific parameters may refer to relevant descriptions for S103˜S105, which will not be described herein again.

In the embodiments of the present application, a plurality of medical sub-images are acquired by performing boundary division on the medical image, and the visual feature classification and the keyword sub-sequence corresponding to each of the medical sub-images are determined respectively, and finally the visual feature vector and the keyword sequence of the medical image are constructed, thereby reducing the data processing volume of the VGG neural network and improving the generation efficiency.

It should be understood that, the sequence numbers of the steps in the above embodiments do not mean the order of execution, and the execution order of each process should be determined by its function and internal logic, and should not constitute any limitation on the implementation process of the embodiments of the present application.

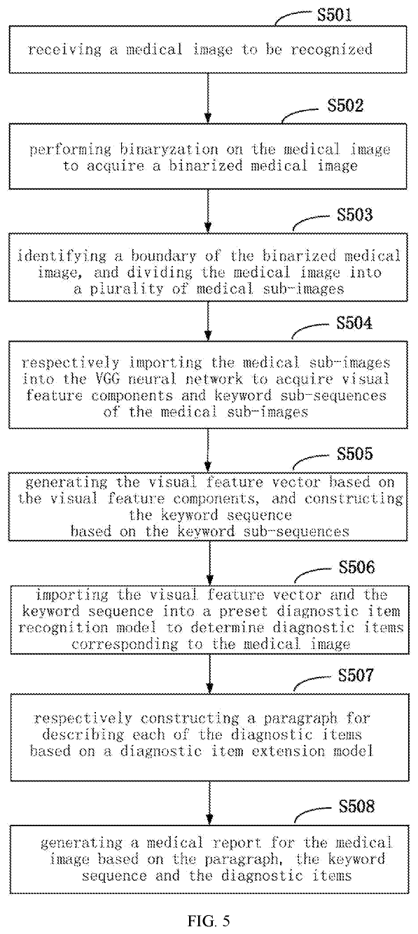

FIG. 6 shows a block diagram of a structure of the device for generating a medical report according to an embodiment of the present application. The device for generating a medical report includes units for performing the steps in the embodiment corresponding to FIG. 1a. For details, please refer to FIG. 1a and related description of the embodiments corresponding to FIG. 1a. For convenience of explanation, only parts related to this embodiment are shown.

Referring to FIG. 6, the device for generating a medical report includes:

a medical image receiving unit 61, configured to receive a medical image to be identified;

a feature vector acquisition unit 62, configured to import the medical image into a preset visual geometric group, such as a VGG neural network, to acquire a visual feature vector and a keyword sequence of the medical image;

a diagnostic item recognition unit 63, configured to import the visual feature vector and the keyword sequence into a preset diagnostic item recognition model to determine a diagnostic item corresponding to the medical image;

a paragraph determination unit 64, configured to construct a paragraph for describing each of the diagnostic items based on the diagnostic item extension model;

a medical report generation unit 65, configured to generate the medical report of the medical image according to the paragraph, the keyword sequence, and the diagnostic item.

Alternatively, the feature vector acquisition unit 62 includes:

a pixel matrix construction unit, configured to construct a pixel matrix of the medical image based on a pixel value of each of pixel points in the medical image and position coordinates of each of pixel values;

a visual feature vector generation unit, configured to perform dimensionality reduction on the pixel matrix through five pooling layers (Maxpools) of the VGG neural network to acquire a visual feature vector;

an index sequence generation unit, configured to import the visual feature vector into a fully connected layer of the VGG neural network, and output an index sequence corresponding to the visual feature vector;

a keyword sequence generation unit, configured to determine a keyword sequence corresponding to the index sequence according to a keyword index table.

Alternatively, the diagnostic item recognition unit 63 includes:

a keyword feature vector construction unit, configured to generate a keyword feature vector corresponding to the keyword sequence based on a sequence number of each of keywords in a preset text corpus;

a preprocessing unit, configured to respectively import the keyword feature vector and the visual feature vector into a preprocessing function to acquire a preprocessed keyword feature vector and a preprocessed visual feature vector; wherein the preprocessing function is specifically as:

$$σ ( {z}_{j} ) = \frac{{e}^{{z}_{j}}}{{Σ}_{i = 1}^{M} {e}^{{z}_{i}}}$$

where, σ(zⱼ) is the value after the j-th element in the keyword feature vector or in the visual feature vector is preprocessed, zⱼ is the value of the j-th element in the keyword feature vector or in the visual feature vector, M is the number of elements corresponding to the keyword feature vector or the visual feature vector;

a preprocessed vector importing unit, configured to use the preprocessed keyword feature vector and the preprocessed visual feature vector as an input of the diagnostic item recognition model, and output a diagnosis item.

Alternatively, the device for generating a medical report further includes:

a training parameter acquisition unit, configured to acquire training visual vectors, training keyword sequences, and training diagnostic items of a plurality of training images;

a learning parameter training unit, configured to use the training visual vectors and the training keyword sequences as an input to a long short-term LSTM neural network, and to use the training diagnostic items as an output of the LSTM neural network, and to adjust each of learning parameters in the LSTM neural network so that the LSTM neural network meets a convergence condition; the convergence condition is as:

$${θ}^{*} = {arg max}_{θ} {\sum }_{S t c} log p ( Visual , Keyword \vert Stc ; θ )$$

where θ* is the adjusted learning parameter, Visual is the training visual vector, Keyword is the training keyword sequence, Stc is the training diagnostic item, p(Visual,Keyword|Stc; θ) represents an output result of a probability value of the training diagnostic item when the training visual vector and the training keyword sequence are imported into the LSTM neural network with the value of the learning parameter is θ, and arg max_{θ} Σ_{Stc} log p(Visual,Keyword|Stc; θ) is the value of the learning parameter when the probability value takes the maximum value;

a unit for generating a diagnostic item recognition model, configured to use the adjusted LSTM neural network as a diagnostic item recognition model.

Alternatively, the device for generating a medical report further includes:

a binarization unit, configured to perform binarization on the medical image to acquire a binarized medical image;

a boundary division unit, configured to identify a boundary of the binarized medical image, and to divide the medical image into a plurality of medical sub-images;

the feature vector acquisition unit 62 includes:

a medical sub-image recognition unit, configured to import each of the medical sub-images into the VGG neural network to acquire visual feature components and keyword sub-sequences of the medical sub-images;

a feature vector combination unit, configured to generate the visual feature vector based on each of the visual feature components, and to form the keyword sequence based on each of the keyword sub-sequences.

Therefore, the device for generating a medical report provided in the embodiments of the present application also does not need to be filled in manually by a doctor, and can automatically output a corresponding medical report according to the features contained in the medical image, which improves the efficiency of generating the medical report, reduces the labor cost, and saves consultation time for the patient.

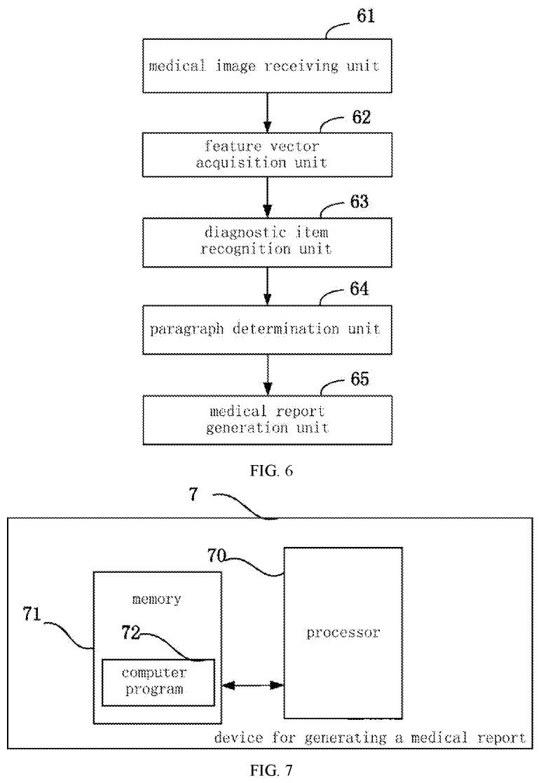

FIG. 7 is a schematic diagram of the device for generating a medical report according to another embodiment of the present application. As shown in FIG. 7, the device 7 for generating a medical report in this embodiment includes a processor 70, a memory 71, and a computer-readable instruction 72 stored in the memory 71 and executable on the processor 70, such as a program for generating a medical report. When executing the computer-readable instruction 72, the processor 70 implements the steps in the above embodiments of the method for generating a medical report, such as the steps of from S101 to S105 as shown in FIG. 1a. Alternatively, when executing the computer-readable instruction 72, the processor 70 implements the function of each of the units in the foregoing device embodiments, such as the functions of the modules 61 to 65 as shown in FIG. 6.

Exemplarily, the computer-readable instruction 72 may be divided into one or more units, and the one or more units are stored in the memory 71 and executed by the processor 70 to complete the present application. The one or more units may be a series of computer-readable instruction segments capable of performing a specific function, and the instruction segments are used to describe an execution process of the computer-readable instruction 72 in the device 7 for generating a medical report. For example, the computer-readable instruction 72 may be divided into a medical image receiving unit, a feature vector acquisition unit, a diagnostic item recognition unit, a description paragraph determination unit, and a medical report generation unit, and the specific functions of the units are described as above.

The device 7 for generating a medical report may be a computing device such as a desktop computer, a notebook, a palmtop computer, or a cloud server or the like. The device for generating a medical report may include, but is not limited to, the processor 70 and the memory 71. Those skilled in the art may understand that FIG. 7 is only an example of the device 7 for generating a medical report and does not constitute a limitation on the device 7 for generating a medical report, which may include more or fewer components than those as shown in the figure, or combine some components or different components. For example, the device for generating a medical report may further include an input device and an output device, a network access device, a bus, and the like.

The processor 70 may be a central processing unit (CPU), or other general-purpose processor, a digital signal processor (DSP), an application specific integrated circuit (ASIC), a field-programmable gate array (FPGA), or other programmable logic device, a discrete gate or a transistor logic device, a discrete hardware component, etc. The general-purpose processor may be a microprocessor or the processor may be any conventional processor or the like.

The memory 71 may be an internal storage unit of the device 7 for generating a medical report, such as a hard disk or a memory of the device 7 for generating a medical report. The memory 71 may also be an external storage device of the device 7 for generating a medical report, such as a plug-in hard disk, a smart media card (SMC), a secure digital (SD) card or a flash card etc. equipped on the device 7 for generating a medical report. Further, the memory 71 may include both an internal storage unit of the device 7 for generating a medical report and an external storage device. The memory 71 is configured to store the computer-readable instruction and other programs and data required by the device for generating a medical report. The memory 71 may also be configured to temporarily store data that has been output or is to be output.

In addition, the various function units in each embodiment of the present application may be integrated into one processing unit, or each of the units may exist separately physically, or two or more units may be integrated into one unit. The above integrated unit may be implemented in a form of hardware or in a form of software function unit.

The above-mentioned embodiments are only used to describe the technical solutions of the present application, but not limited to the present application. Although the present application has been described in detail with reference to the foregoing embodiments, those skilled in the art should understand that the technical solutions described in foregoing embodiments can still be modified, or some of the technical features may be equivalently substituted. These modifications or substitutions do not deviate the essence of the corresponding technical solutions from the spirit and scope of the technical solutions of the embodiments of the present application, and should be included within the scope of the present application.

1. A method for generating a medical report, comprising:
receiving a medical image to be recognized;
importing the medical image into a preset VGG neural network to acquire a visual feature vector and a keyword sequence of the medical image;
importing the visual feature vector and the keyword sequence into a preset diagnostic item recognition model to determine diagnostic items corresponding to the medical image;
respectively constructing a paragraph for describing each of the diagnostic items based on a diagnostic item extension model;
generating a medical report for the medical image based on the paragraph, the keyword sequence and the diagnostic items.

2. The method according to claim 1, wherein the step of importing the medical image into a preset VGG neural network to acquire a visual feature vector and a keyword sequence of the medical image comprises:
constructing a pixel matrix of the medical image based on pixel values of pixels in the medical image and position coordinates of the pixel values;
performing dimensionality reduction on the pixel matrix through five pooling layers of the VGG neural network to acquire the visual feature vector;
importing the visual feature vector into a fully connected layer of the VGG neural network and outputting an index sequence corresponding to the visual feature vector;
determining the keyword sequence corresponding to the index sequence according to a keyword index table.

3. The method according to claim 1, wherein the step of importing the visual feature vector and the keyword sequence into a preset diagnostic item recognition model to determine diagnostic items corresponding to the medical image comprises:
generating a keyword feature vector corresponding to the keyword sequence based on sequence numbers of keywords in a preset text corpus;
respectively importing the keyword feature vector and the visual feature vector into a preprocessing function to acquire a preprocessed keyword feature vector and a preprocessed visual feature vector; wherein the preprocessing function is specifically as:
$$σ ( {z}_{j} ) = \frac{{e}^{{z}_{j}}}{{Σ}_{i = 1}^{M} {e}^{{z}_{i}}}$$
where σ(zⱼ) is a value of j-th element in the preprocessed keyword feature vector or in the preprocessed visual feature vector, zⱼ is a value of j-th element in the keyword feature vector or in the visual feature vector, M is the number of elements corresponding to the keyword feature vector or the visual feature vector;
determining the preprocessed keyword feature vector and the preprocessed visual feature vector as an input of the diagnostic item recognition model, and outputting the diagnostic items.

4. The method according to claim 1, wherein the method further comprises:
acquiring training visual vectors, training keyword sequences and training diagnostic items of a plurality of training images;
determining the training visual vectors and the training keyword sequences as an input of a LSTM neural network, determining the training diagnostic items as an output of the LSTM neural network, and adjusting learning parameters in the LSTM neural network so that the LSTM neural network meets a convergence condition; wherein the convergence condition is as:
$${θ}^{*} = {arg max}_{θ} {\sum }_{S t c} log p ( Visual , Keyword \vert Stc ; θ )$$
where θ* is the adjusted learning parameter, Visual is the training visual vector, Keyword is the training keyword sequence, Stc is the training diagnostic item, p(Visual, Keyword|Stc; θ) represents an output result of a probability value of the training diagnostic item when the training visual vector and the training keyword sequence are imported into the LSTM neural network with the value of the learning parameter is θ, and arg max_{θ} Σ_{Stc} log p(Visual,Keyword|Stc; θ) is the value of the learning parameter when the probability value takes a maximum value;
determining the adjusted LSTM neural network as the diagnostic item recognition model.

5. The method according to claim 1, wherein, after receiving a medical image to be recognized, the method further comprises:
performing binaryzation on the medical image to acquire a binarized medical image;
identifying a boundary of the binarized medical image, and dividing the medical image into a plurality of medical sub-images;
wherein the step of importing the medical image into a preset VGG neural network to acquire a visual feature vector and a keyword sequence of the medical image comprises:
respectively importing the medical sub-images into the VGG neural network to acquire visual feature components and keyword sub-sequences of the medical sub-images;
generating the visual feature vector based on the visual feature components, and constructing the keyword sequence based on the keyword sub-sequences.

6-10. (canceled)

11. A device for generating a medical report, comprising a memory, a processor, and a computer-readable instruction stored in the memory and executable on the processor, wherein the processor, when executing the computer-readable instruction, implements the following steps of:
receiving a medical image to be recognized;
importing the medical image into a preset VGG neural network to acquire a visual feature vector and a keyword sequence of the medical image;
importing the visual feature vector and the keyword sequence into a preset diagnostic item recognition model to determine diagnostic items corresponding to the medical image;
constructing a paragraph for describing each of the diagnostic items respectively based on a diagnostic item extension model;
generating a medical report for the medical image based on the paragraph, the keyword sequence and the diagnostic items.

12. The device according to claim 11, wherein the step of importing the medical image into a preset VGG neural network to acquire a visual feature vector and a keyword sequence of the medical image comprises:
constructing a pixel matrix of the medical image based on pixel values of pixels in the medical image and position coordinates of the pixel values;
performing dimensionality reduction on the pixel matrix through five pooling layers of the VGG neural network to acquire the visual feature vector;
importing the visual feature vector into a fully connected layer of the VGG neural network and outputting an index sequence corresponding to the visual feature vector;
determining the keyword sequence corresponding to the index sequence according to a keyword index table.

13. The device according to claim 12, wherein the step of importing the visual feature vector and the keyword sequence into a preset diagnostic item recognition model to determine diagnostic items corresponding to the medical image comprises:
generating a keyword feature vector corresponding to the keyword sequence based on sequence numbers of keywords in a preset text corpus;
respectively importing the keyword feature vector and the visual feature vector into a preprocessing function to acquire a preprocessed keyword feature vector and a preprocessed visual feature vector; wherein the preprocessing function is specifically as:
$$σ ( {z}_{j} ) = \frac{{e}^{{z}_{j}}}{{Σ}_{i = 1}^{M} {e}^{{z}_{i}}}$$
where σ(zⱼ) is a value of j-th element in the preprocessed keyword feature vector or in the preprocessed visual feature vector, zⱼ is a value of j-th element in the keyword feature vector or in the visual feature vector, M is the number of elements corresponding to the keyword feature vector or the visual feature vector;
determining the preprocessed keyword feature vector and the preprocessed visual feature vector as an input of the diagnostic item recognition model, and outputting the diagnostic items.

14. The device according to claim 11, wherein the processor, when executing the computer-readable instruction, further implements the following steps of:
acquiring training visual vectors, training keyword sequences and training diagnostic items of a plurality of training images;
determining the training visual vectors and the training keyword sequences as an input of a LSTM neural network, determining the training diagnostic items as an output of the LSTM neural network, and adjusting learning parameters in the LSTM neural network so that the LSTM neural network meets a convergence condition; wherein the convergence condition is as:
$${θ}^{*} = {arg max}_{θ} {\sum }_{S t c} log p ( Visual , Keyword \vert Stc ; θ )$$
where θ* is the adjusted learning parameter, Visual is the training visual vector, Keyword is the training keyword sequence, Stc is the training diagnostic item, p(Visual, Keyword|Stc; θ) represents an output result of a probability value of the training diagnostic item when the training visual vector and the training keyword sequence are imported into the LSTM neural network with the value of the learning parameter is θ, and arg max_{θ} Σ_{Stc} log p(Visual,Keyword|Stc; θ) is the value of the learning parameter when the probability value takes a maximum value;
determining the adjusted LSTM neural network as the diagnostic item recognition model.

15. The device according to claim 11, wherein, after receiving a medical image to be recognized, the processor, when executing the computer-readable instruction, further implements the following steps of:
performing binaryzation on the medical image to acquire a binarized medical image;
identifying a boundary of the binarized medical image, and dividing the medical image into a plurality of medical sub-images;
wherein the step of importing the medical image into a preset VGG neural network to acquire a visual feature vector and a keyword sequence of the medical image comprises:
importing the medical sub-images into the VGG neural network respectively to acquire visual feature components and keyword sub-sequences of the medical sub-images;
generating the visual feature vector based on the visual feature components, and constructing the keyword sequence based on the keyword sub-sequences.

16. A computer readable storage medium, stored with a computer readable instruction, wherein the computer readable instruction, when executed by a processor, implements the following steps of:
receiving a medical image to be recognized;
importing the medical image into a preset VGG neural network to acquire a visual feature vector and a keyword sequence of the medical image;
importing the visual feature vector and the keyword sequence into a preset diagnostic item recognition model to determine diagnostic items corresponding to the medical image;
constructing a paragraph for describing each of the diagnostic items respectively based on a diagnostic item extension model;
generating a medical report for the medical image based on the paragraph, the keyword sequence and the diagnostic items.

17. The computer readable storage medium according to claim 16, wherein the step of importing the medical image into a preset VGG neural network to acquire a visual feature vector and a keyword sequence of the medical image comprises:
constructing a pixel matrix of the medical image based on pixel values of pixels in the medical image and position coordinates of the pixel values;
performing dimensionality reduction on the pixel matrix through five pooling layers of the VGG neural network to acquire the visual feature vector;
importing the visual feature vector into a fully connected layer of the VGG neural network and outputting an index sequence corresponding to the visual feature vector;
determining the keyword sequence corresponding to the index sequence according to a keyword index table.

18. The computer readable storage medium according to claim 16, wherein the step of importing the visual feature vector and the keyword sequence into a preset diagnostic item recognition model to determine diagnostic items corresponding to the medical image comprises:
generating a keyword feature vector corresponding to the keyword sequence based on sequence numbers of keywords in a preset text corpus;
respectively importing the keyword feature vector and the visual feature vector into a preprocessing function to acquire a preprocessed keyword feature vector and a preprocessed visual feature vector; wherein the preprocessing function is specifically as:
$$σ ( {z}_{j} ) = \frac{{e}^{{z}_{j}}}{{Σ}_{i = 1}^{M} {e}^{{z}_{i}}}$$
where σ(zⱼ) is a value of j-th element in the preprocessed keyword feature vector or in the preprocessed visual feature vector, zⱼ is a value of j-th element in the keyword feature vector or in the visual feature vector, M is the number of elements corresponding to the keyword feature vector or the visual feature vector;
determining the preprocessed keyword feature vector and the preprocessed visual feature vector as an input of the diagnostic item recognition model, and outputting the diagnostic items.

19. The computer readable storage medium according to claim 16, wherein the computer readable instruction, when executed by the processor, further implements the following steps of:
acquiring training visual vectors, training keyword sequences and training diagnostic items of a plurality of training images;
determining the training visual vectors and the training keyword sequences as an input of a LSTM neural network, determining the training diagnostic items as an output of the LSTM neural network, and adjusting learning parameters in the LSTM neural network so that the LSTM neural network meets a convergence condition; wherein the convergence condition is as:
$${θ}^{*} - {arg max}_{θ} {\sum }_{S t c} log p ( Visual , Keyword \vert Stc ; θ )$$
where θ* is the adjusted learning parameter, Visual is the training visual vector, Keyword is the training keyword sequence, Stc is the training diagnostic item, p(Visual,Keyword|Stc; θ) represents an output result of a probability value of the training diagnostic item when the training visual vector and the training keyword sequence are imported into the LSTM neural network with the value of the learning parameter is θ, and arg max_{θ} Σ_{Stc} log p(Visual,Keyword|Stc; θ) is the value of the learning parameter when the probability value takes a maximum value;
determining the adjusted LSTM neural network as the diagnostic item recognition model.

20. The computer readable storage medium according to claim 16, wherein, after receiving a medical image to be recognized, the computer readable instruction, when executed by the processor, further implements the following steps of:
performing binaryzation on the medical image to acquire a binarized medical image;
identifying a boundary of the binarized medical image, and dividing the medical image into a plurality of medical sub-images;
wherein the step of importing the medical image into a preset VGG neural network to acquire a visual feature vector and a keyword sequence of the medical image comprises:
importing the medical sub-images into the VGG neural network respectively to acquire visual feature components and keyword sub-sequences of the medical sub-images;
generating the visual feature vector based on the visual feature components, and constructing the keyword sequence based on the keyword sub-sequences.
